(19) United States
(12) Patent Application Publication
(10) Pub. No.: US 2006/0124494 A1
(43) Pub. Date: Jun. 15, 2006

(54) Shape retaining flexible package with easy access opening feature

(75) Inventors: James Joseph Clark JR., Appleton, WI (US); Jacqueline Marie Maxton, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/010,109

(22) Filed: Dec. 9, 2004

(51) Int. Cl. A61B 19/02; B65D 30/00; B65D 33/00

(52) U.S. Cl. 206/440; 383/207; 383/127

(57) ABSTRACT

The present invention provides a shape retaining flexible package having a top surface, a bottom surface and at least one side wall. The top surface and the bottom surface each being directly or indirectly connected to the at least one side wall and form an interior compartment capable of containing a plurality of articles. The top surface of the package has a width and a length. The package also has an opening device located on the top surface. The opening device has at least one line of weakness and the opening device is confined to the top surface such that there is a portion of the top surface located between the opening device and an edge created by the direct or indirect connection of the top surface to the at least one side wall. The opening device has a width at some point along the length of the opening device which is between about 40% and about 99% of the width of the top surface and a length at some point along the width of the opening device which is between about 40% and about 99% of the length of the top surface. The opening device provides for easy opening of the flexible package and provides for easy access to articles within the interior compartment of the flexible package. In addition, the flexible package retains its shape even as the articles are removed from the flexible package.

### FIELD OF THE INVENTION

The present invention relates to a flexible package for containing and dispensing articles. More particularly, the present invention relates to a flexible packaging bag having an easy-open feature, which allows for easy access to the articles contained within the bag.

### BACKGROUND OF THE INVENTION

Packaging bags made from flexible polymeric materials have been used for packaging various types of products, including, for example, adult incontinence articles, diapers, training pants, feminine care products, among many other items. These bags provide packaging for the products, creating a carton-like look and configuration which facilitates display of the products on the retail shelf for consumers to purchase. These bags also provide a convenient way for the consumer to transport the products from the retailer to the consumer's home or place of use. Typically, these bags are provided with an opening device that allows the consumer to access products contained within the bags.

Currently available opening devices often compromise the structure of the bag, causing the bag to fully or partially collapse on itself and any product remaining in the bag as the products are removed. This can make it difficult for the consumer to remove remaining product from the bag. Further, when the bag collapses, the bag looses its carton-like structure, which is often considered by consumers to lack neatness. On other currently available bags, the opening device is not of a sufficient size to easily access and remove the products from the bag. If the opening device is not of a sufficient size, then extra effort may be required from a consumer to remove the product from the bag. In addition, many of currently available bag opening devices are often difficult for consumers to open for one reason or another. Many opening devices have a pinch and pull opening means. It is widely recognized that grasping or pinching a packaging bag to open the bag can be difficult for older users and those users with diseases which affect the dexterity of a user's hands, such as arthritis.

To avoid the problems described above, there is a need in the art for a flexible packaging bag which retains its shape after opening and provides easy access to the articles stored inside the bag to facilitate easy removal of the articles from the bag. In addition, there is a need in the art for a flexible packaging bag which is easy for consumers to open, particularly for consumers who have difficulty in grasping or pinching materials such as polymer films.

### SUMMARY OF THE INVENTION

Generally stated, the present invention provides a shape retaining flexible package having a top surface, a bottom surface and at least one side wall. The top surface and the bottom surface each being directly or indirectly connected to the at least one side wall and form an interior compartment capable of containing a plurality of articles. The top surface of the package has a width and a length. The package also has an opening device located on the top surface. The opening device has at least one line of weakness and the opening device is confined to the top surface such that there is a portion of the top surface located between the opening device and an edge created by the direct or indirect connection of the top surface to the at least one side wall. The opening device has a width at some point along the length of the opening device which is between about 40% and about 99% of the width of the top surface and a length at some point along the width of the opening device which is between about 40% and about 99% of the length of the top surface. The opening device provides for easy opening of the flexible package and provides for easy access to articles within the interior compartment of the flexible package.

In another aspect of the present invention, the present invention provides a shape retaining flexible package having a top surface, a bottom surface and at least one side wall. The top surface and the bottom surface each being directly or indirectly connected to the at least one side wall and form an interior compartment capable of containing a plurality of articles. The top surface of the package has a width and a length. An opening device is located on the top surface. The opening device allows for the package to be opened, revealing an opening in which articles contained within the package may be accessed. The opening is confined to the top surface such that there is a portion of the top surface located between the opening and an edge created by the direct or indirect connection of the top surface to the at least one side wall. The opening has a width at some point along the length of the opening which is between about 40% and about 99% of the width of the top surface and a length at some point along the width of the opening which is between about 40% and about 99% of the length of the top surface. The opening provides for easy access to articles within the interior compartment of the flexible package.

In further aspect of the present invention, the flexible package has a front wall, a back wail, a first side wail and a second side wail, wherein the front wail is opposed to the back wall and first side wall is opposed the second side wall. The front wall and the back wall are directly or indirectly connected to the first and second side walls as well as the top surface and the bottom surface of the flexible package.

In an additional aspect of the present invention, the flexible package of the present invention may have a plurality of articles contained within the interior compartment of the flexible package. The articles may be arranged in the flexible package in a single row or may be arranged in at least two rows, each row comprising at least a portion of the plurality of articles.

In yet another aspect of the present invention, the articles within the interior compartment may be arranged perpendicular to the top surface. These articles may be personal care articles, in particular, absorbent personal care articles.

### BRIEF DESCRIPTION OF THE DRAWINGS

FIG. 1 shows a perspective view of a flexible package of the present invention having an opening device in an unopened state.

FIG. 2 shows a perspective view of a flexible package of the present invention having an opening device in an opened state revealing the articles within the flexible package.

FIG. 3 shows a plane front view of a flexible package of the present invention depicting two horizontal rows of articles stacked within the package.

FIG. 4 shows a bottom view of the flexible package of the present invention depicting a bottom seal.

FIG. 5 shows a top view of the flexible package of the present invention with an alternative opening device configuration.

FIG. 6 shows a top view of the flexible package of the present invention with an alternative opening device configuration.

FIG. 7 shows a top view of the flexible package of the present invention with an opening device configuration with an opening aid.

FIG. 8 shows a top view of the flexible package of the present invention with an opening device configuration with an alternative opening aid.

FIG. 9 shows a top view of the flexible package of the present invention with an opening device configuration with an alternative opening aid.

FIG. 10 shows a top view of the flexible package of the present invention with an alternative opening device configuration.

FIG. 11 shows a top view of the flexible package of the present invention with an alternative opening device configuration.

FIG. 12 shows a perspective view of the flexible package of the present invention with the opening device removed and a message on the reverse side of the opening device.

FIG. 13 shows a perspective view of the flexible package of the present invention with a preformed opening and a covering device.

FIG. 14 shows a perspective view of the flexible package of the present invention with a preformed opening and another covering device in a closed position.

FIG. 15 shows a perspective view of the flexible package of the present invention with a preformed opening or opening device and covering device shown in FIG. 14, with the covering device in an opened position.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “line of weakness” as used herein, refers to any region or area of weakened material, preferably having a length, but not necessarily a defined width. A “line of weakness” can include linear and non-linear patterns, such as curvilinear patterns of weakness, or other shapes, such as circles, rectangles, and so forth. A line of weakness includes a perforation or other series of cuts, a thinning or breakage or separation of material, or a strip of a different kind of material bridging between adjacent portions of material that is more easily torn or broken than the adjacent portions, and which allows the user or manufacturer to separate the adjacent portions along the line of weakness. A line of weakness can further include a single extended slit or cut.

As used herein, the terminology such as “vertical”, “horizontal”, “top”, “bottom”, “front”, “back”, “end” and “sides” are referenced according to the views presented. It should be understood, however, that the terms are used only for purposes of description, and are not intended to be used as limitations. Accordingly, orientation of an object or a combination of objects may change without departing from the scope of the invention. As a point of reference for the claims and in the present specification, the term “top” refers to a panel or side of the package with an opening device or opening.

As used herein, the term “opening device” refers to the area of the package which can be manipulated by a user to access any articles contained within the flexible package. The opening device may be removable from the package or may remain attached to the package after the opening device is opened. The opening device may be a part of the top surface or a separate material attached to the top surface.

As used herein, the term “opening” refers to a removed or otherwise cut-away portion of the flexible package which allows access to the articles contained within the interior compartment and allows for the articles to be removed from the interior compartment.

It should be understood that the terms “personal care product” or “personal care article” as used herein refers to any article used to control bodily fluids, and includes “absorbent products,” or “absorbent articles” which refers to any article configured to absorb and retain bodily exudates, including urine, bowel movements, blood and menses, and includes such a product in a packaged and unpackaged configuration. As such, personal care products, as used herein, includes without limitation, diapers, child toilet training pants, adult incontinence garments, male incontinence products, tampons, vaginal suppositories, pantiliners, pads, sanitary napkins, tissues, wipes, etc. Examples of commercially available personal care products include, without limitation, Poise@ feminine care products, including pantiliners and pads, and Kotex® feminine care products, including pads, tampons and liners, all available from Kimberly-Clark Corporation, Neenah, Wis.

As used herein, the term “shape retaining” is intended to mean that the shape of the flexible package with articles contained therein is essentially the same as the shape of the package while the articles are being removed and with all of the articles removed from the interior compartment of the package. That is the sides of the package do not tend to fall inward towards the interior compartment or outward away from the interior compartment and the top surface does not fall inward towards the interior compartment. It is noted that a small degree, i.e., less than about a 20% variation in width and/or length of the top surface, comparing the length or width of the top surface while the package is full to the length or width of the top surface with the articles removed, is considered to be shape retaining. For example, if the package has a width of 10 cm at the top surface with the articles contained therein, if the package has a width at the same point of 8-12 cm after the articles are removed, the package is considered to be shape retaining.

As used herein, the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configuration of the material. These configurations include, but are not limited to isotactic, syndiotactic and random symmetries.

### DETAILED DESCRIPTION OF THE INVENTION

In the following detailed description of the present invention, reference is made to the accompanying drawings which form a part hereof, and in which is shown by way of illustration specific embodiments in which the inventions may be practiced. These embodiments are described in sufficient detail to enable those skilled in the art to practice the invention, and it is to be understood that other embodiments may be utilized and that mechanical, procedural, and other changes may be made without departing from the spirit and scope of the present invention. The following detailed description is, therefore, not to be taken in a limiting sense, and the scope of the present invention is defined only by the appended claims, along with the full scope of equivalents to which such claims are entitled.

Generally stated, the present invention provides a shape retaining flexible package having a top surface, a bottom surface and at least one side wall. The top surface and the bottom surface each being directly or indirectly connected to the at least one side wall and form an interior compartment capable of containing a plurality of articles. The top surface of the package has a width and a length. The package also has an opening device located on the top surface. The opening device has at least one line of weakness and the opening device is confined to the top surface such that there is a portion of the top surface located between the opening device and an edge created by the direct or indirect connection of the top surface to the at least one side wall. The opening device has a width at some point along the length of the opening device which is between about 40% and about 99% of the width of the top surface and a length at some point along the width of the opening device which is between about 40% and about 99% of the length of the top surface. The opening device provides for easy opening of the flexible package and provides for easy access to articles within the interior compartment of the flexible package.

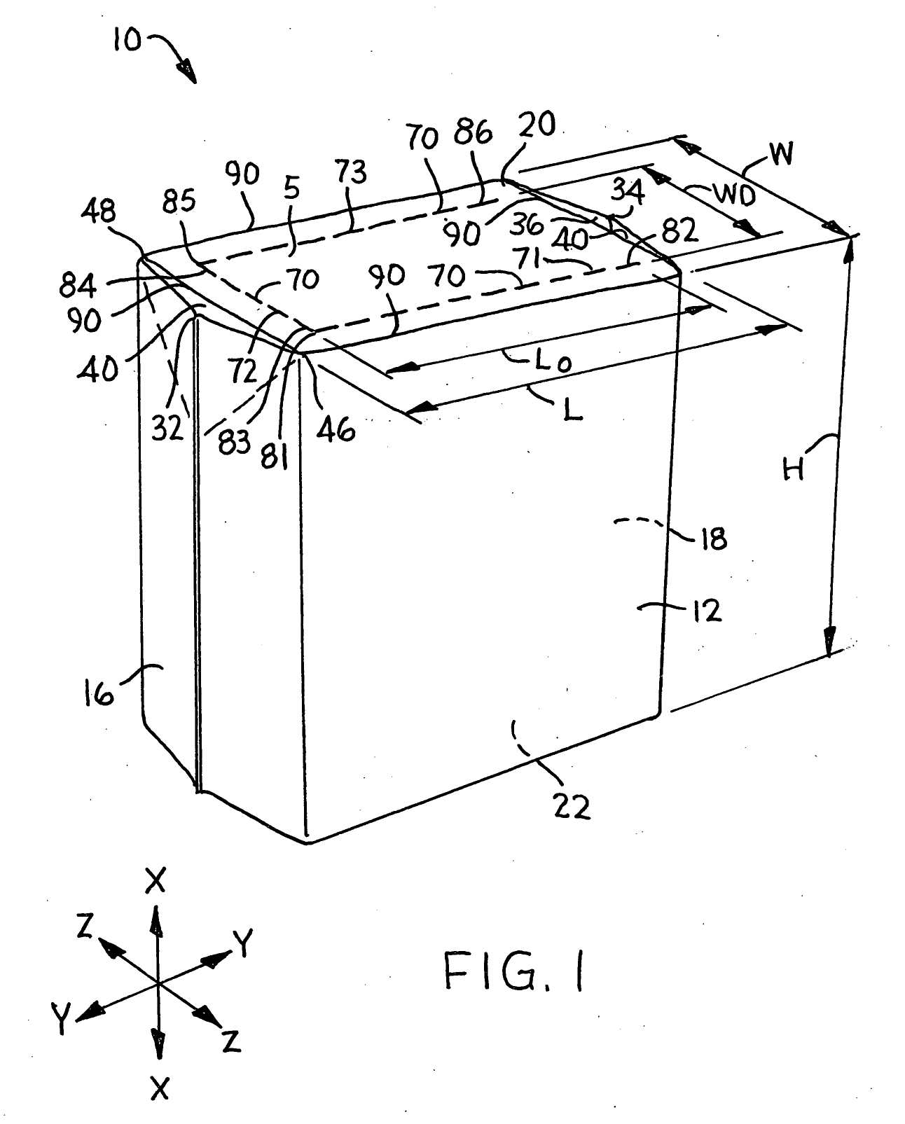

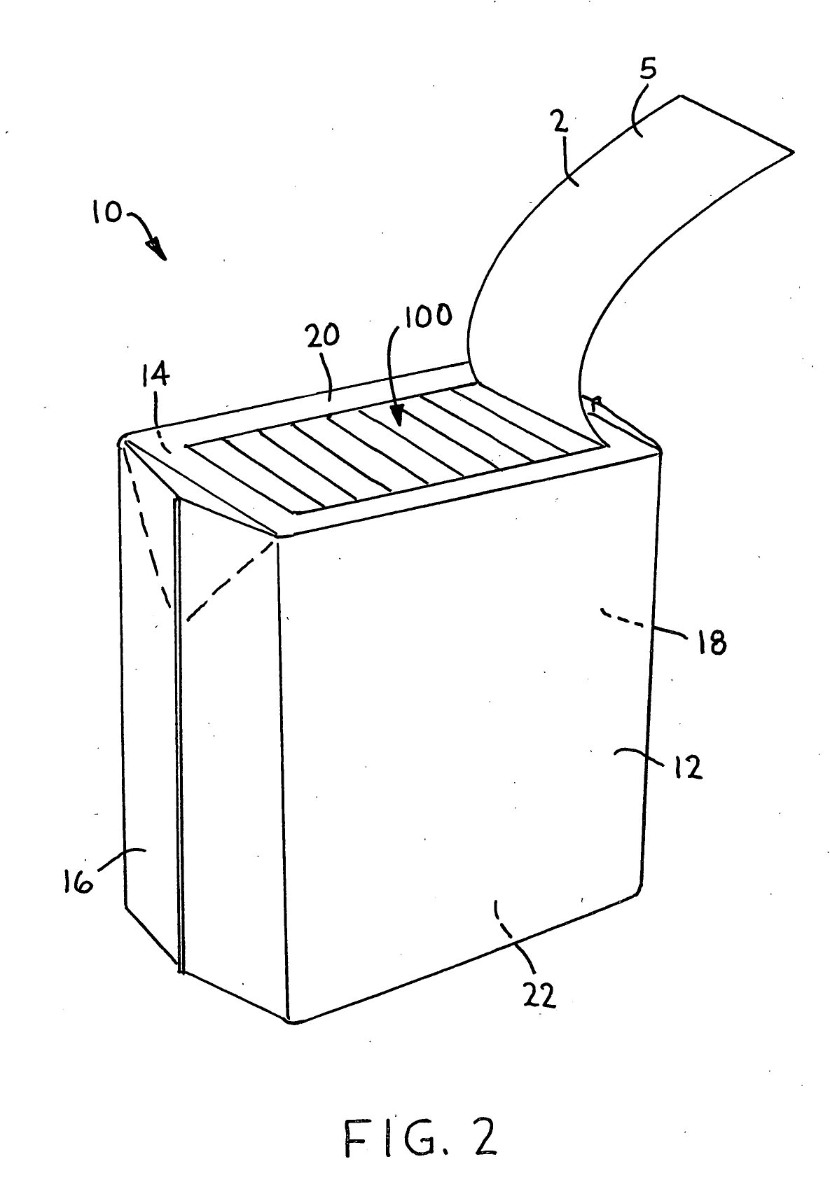

To obtain a better understanding of the present invention, attention is directed to FIGS. 1-12. As is shown in FIGS. 1, 2 and 3, a flexible package 10 having an opening device 5 is provided. As shown in FIGS. 1 and 2, the flexible package has a top surface 20, a bottom surface 22, a front wall 12, a back wall 14, and a first side wall 16 and a second side wall 18. The first side wall 16 and the second side wall 18 are opposed to each other; as are the front wall 12 and the back wall 14, and the top surface 20 and the bottom surface 22. As shown, the front wall 12, the back wall 14, the two side walls 16 and 18, the top surface 20 and the bottom surface 22 are directly or indirectly connected together to form the package 10 which has an interior compartment 24 with an interior volume. The interior compartment 24 is capable of containing a plurality of articles 26. The package 10 has a longitudinal axis Y-Y, a transverse axis Z-Z and a vertical axis X-X. The package also has a height H, a width (depth) W and a length L. As is shown in FIGS. 1 and 2, the flexible package has four side walls. It is not outside the present invention that the flexible package has a single side wall which is continuous, two side walls, three side walls or more than four side walls. It is noted that in a common configuration, the flexible package has four side walls and for purposes of understanding the present invention only, the flexible package will be described in terms of a flexible package having four side walls.

In the present invention, the package 10 has an opening device 5 located on the top surface 20. The opening device 5 is formed from at least one line of weakness 70 on the top surface 20. This line of weakness 70 is confined to the top surface 20 such that there is a portion of the top surface located between the opening device 5 and an edge 90 created by the direct or indirect connection of the top surface 20 to the at least one side wall. Stated another way, the opening device 5 does not extend beyond the top surface 20 to any of the side walls. In addition, the opening device has a width Wo at a widest point along the length Lo of the opening device which is between about 40% and about 99% of the width W of the top surface 20. Further, the opening device has a length Lo at a longest point along the width Wo of the opening device which is between about 40% and about 99% of the length L of the top surface 20. If the width of the opening device Wo at its widest point is less than about 40% of the width W of the top surface 20 or the length of the opening device Lo at its longest point is less than about 40% of the length L of the top surface, it can be difficult to remove the articles contained within the interior compartment 24. On the other hand, if the width of the opening device Wo is greater than about 99% of the width W of the top surface or the length of the opening device Lo is greater than about 99% of the length L of the top surface, the package will tend not to retain its shape, making the package appear being untidy to a consumer. When the flexible package does not retain its shape, it can be difficult to remove an article 26 from the interior compartment 24, since the package may collapse on itself and any articles 26 contained within the interior compartment 24. In one embodiment, the opening device has a width Wo at its widest point along the length Lo of the opening device which is between about 50% and about 90% of the width W of the top surface. Desirably, the width Wo at its widest point along the length Lo of the opening device is between about 60% and about 80% of the width W of the top surface. In a similar manner, the opening device has a length Lo at its longest point along the width Wo of the opening device is between about 50% and about 90% of the length L of the top surface. Desirably, the length Lo at its widest point along the width Wo of the opening device that is between about 60% and about 80% of the length L of the top surface. The opening device 5, when opened, forms an opening 100 in the top surface which allows a consumer to access the articles 26 within the interior compartment 24 of the flexible package.

In the present invention, for the width W and length L of the top surface 20 are measured as follows. The width W is the widest distance between the front wall 12 and the back wall 14 measured along the transverse axis Z-Z. In a similar manner, the length L of the top surface is the longest distance between the first side wall 16 and the second side wall 18, measured along the longitudinal axis Y-Y. Generally, the widest distance will occur near a midpoint of the top surface 20 along the length L or at the ends of the length L near the first and/or second sides. Similarly, the longest distance will generally occur near a midpoint of the top surface 20 along the width W or at the ends of the width W, near the front and/or back wall.

The line of weakness 70 which defines the opening device can be formed by any means, which will facilitate separation of the opening device 5 from the top surface 20 along the lines of weakness. An example of a line of weakness includes, without limitation, a perforated line, a line formed by a plurality of openings, such as slots separated by a plurality of land areas, a line of reduced material thickness, a weakened line formed by joining two sections of material together, or any other structural configuration known to those skilled in the art. Each of these lines of weakness are known to those skilled in the art and can be formed by known methods including, for example, mechanical treating of the package material, such as forming perforations in the package material.

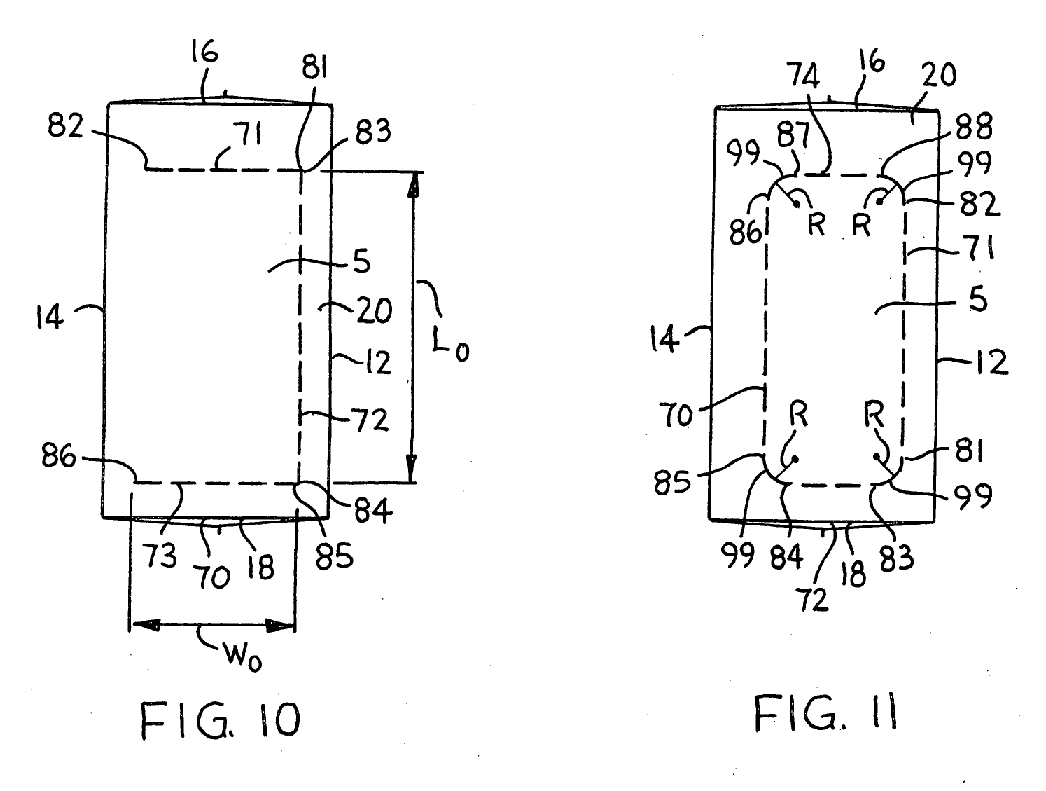

Referring to FIGS. 1, 2, 5, 6, 7, 8, 9, 10 and 11, different configurations of the opening device 5 are shown. In FIGS. 1, 9 and 10, the opening device 5 has three lines of weakness 70, including the first line of weakness 71, the second line of weakness 72 and the third line of weakness 73. The first line of weakness 71 has a first end 81 and a second end 82, the second line of weakness 72, has a first end 83 and a second end 84, and the third line of weakness 73 has a first end 85 and a second end 86. The first end 81 of the first line of weakness 71 is adjacent the first end 83 of the second line of weakness 72, and the second end 84 of the second line of weakness 72 is adjacent the first end 85 of the third line of weakness 73. In the configuration shown in FIGS. 1 and 10, the first line of weakness 71 is parallel to the third line of weakness 73 and the second line of weakness 72 is perpendicular to both the first and third lines of weakness 71 and 73. In the configuration shown in FIG. 9, the first and third lines of weakness 71 and 73 are non-linear, while the second line of weakness 72 is linear. In addition, in the configurations of FIGS. 1, 9 and 10, the opening device 5 remains connected or attached to the flexible package 10 after opening, as is shown in FIG. 2.

In FIG. 6, the opening device 5 has three lines of weakness 70, wherein one of the lines of weakness is non-linear and two are linear, the lines of weakness including a first line of weakness 71, a second line of weakness 72 and a third line of weakness 73. The first line of weakness 71 has a first end 81 and a second end 82, the second line of weakness 72, has a first end 83 and a second end 84, and the third line of weakness has a first end 85 and a second end 86. The first end 81 of the first line of weakness 71 is adjacent the first end 83 of the second line of weakness 72, the second end 84 of the second line of weakness 72 is adjacent the first end 85 of the third line of weakness 73, and the second end 81 of the first line of weakness is adjacent the second end 86 of the third line of weakness 73. In the configuration shown in FIG. 6, the opening device 5 is removable from the flexible package 10.

FIG. 7 shows an opening device having four lines of weakness, including a first line of weakness 71, a second line of weakness 72, a third line of weakness 73 and a fourth line of weakness 74. The first line of weakness 71 has a first end 81 and a second end 82, the second line of weakness 72, has a first end 83 and a second end 84, the third line of weakness has a first end 85 and a second end 86 and the fourth line of weakness has a first end 87 and a second end 88. The first end 81 of the first line of weakness 71 is adjacent the first end 83 of the second line of weakness 72, the second end 84 of the second line of weakness 72 is adjacent the first end 85 of the third line of weakness 73, the second end 86 of the third line of weakness is adjacent the first end 87 of the fourth line of weakness 74 and the second end 82 of the first line of weakness 71 is adjacent the second end 88 of the fourth line of weakness 74. In the configuration shown in FIG. 7, the first line of weakness 71 is parallel to the third line of weakness 73 and the second line of weakness 72 and fourth line of weakness 74 are perpendicular to both the first and third lines of weakness 71 and 73. As a result, the second line of weakness 72 is parallel to the fourth line of weakness 74.

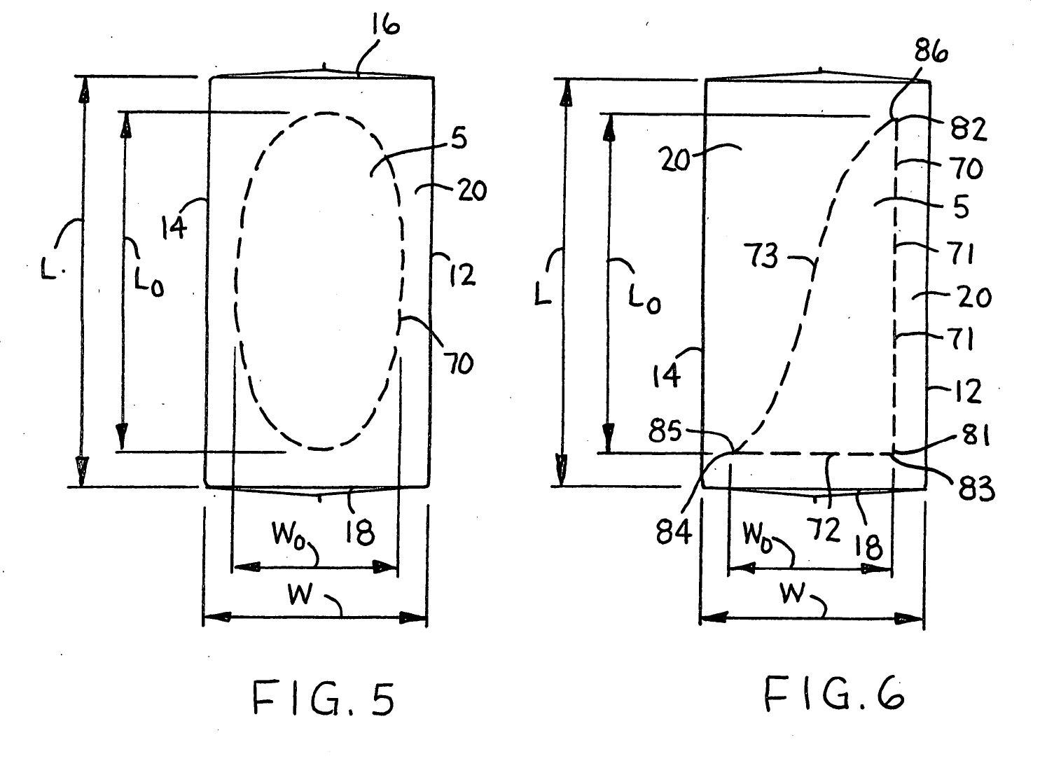

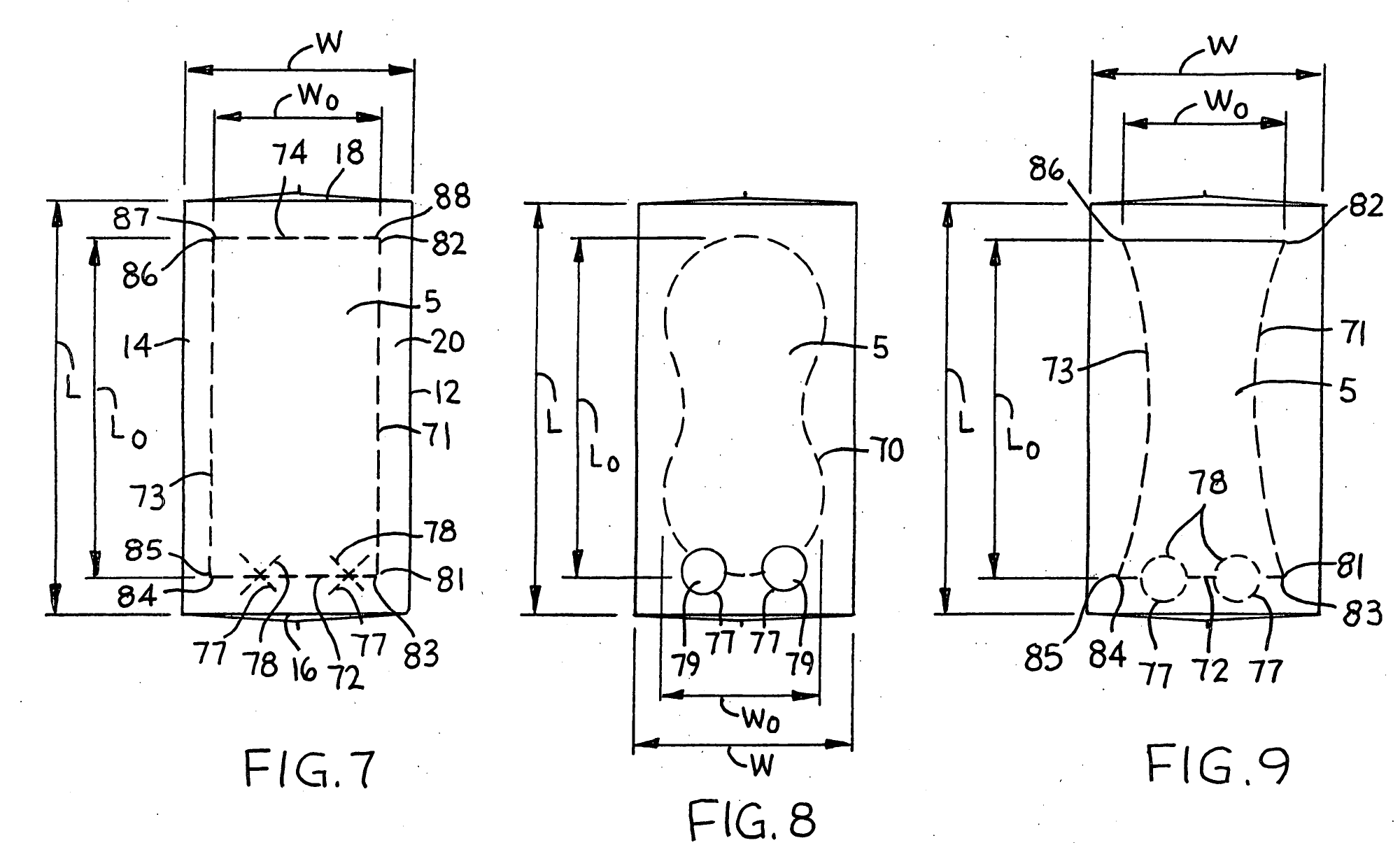

In FIGS. 5 and 8, the opening device is a single continuous line of weakness 70 having an oval shape (FIG. 5) or a bilobal shape (FIG. 8). The shape of the opening device is not critical to the present invention, so long as the width Wo and length Lo conditions mentioned above are met. Shapes and configurations other than those shown in the figures may be used without departing from the scope of the present invention.

In FIGS. 1, and 6-10, the end of one line of weakness is adjacent to the end of another line of weakness, and is shown in these figures to be essentially the same point. However, it is not necessary in the present invention for the end of one line of weakness to be the same point or essentially the same point as the end of another line of weakness. In this case, the end points of the lines of weakness should be sufficiently close together such that the opening device may be opened without requiring much additional force to open the opening device. Optionally, the end of one line of weakness may be joined to the end of another line of weakness by a connecting line. In this regard, attention is directed to FIG. 11, which shows an opening device having four lines of weakness, including a first line of weakness 71, a second line of weakness 72, a third line of weakness 73 and a fourth line of weakness 74. The first line of weakness 71 has a first end 81 and a second end 82, the second line of weakness 72, has a first end 83 and a second end 84, the third line of weakness has a first end 85 and a second end 86 and the fourth line of weakness has a first end 87 and a second end 88. The first end 81 of the first line of weakness 71 is adjacent the first end 83 of the second line of weakness 72, the second end 84 of the second line of weakness 72 is adjacent the first end 85 of the third line of weakness 73, the second end 86 of the third line of weakness is adjacent the first end 87 of the fourth line of weakness 74 and the second end 82 of the first line of weakness 71 is adjacent the second end 88 of the fourth line of weakness 74. As is also shown in FIG. 11, the end of each line of weakness is joined to the end of another line of weakness using a connecting line 99. The connecting line 99 may be a straight line, an arc, a zig-zag line or the combination of all three types of line. Other connecting lines may be used without departing from the scope of the present invention. If the connecting line 99 is an arch, the arc will have a radius r. The radius of the arc is not important to the present invention, but the radius is generally less than about 5 inches (12.7 cm), but the radius selected may be adjusted, depending on the size of the opening and the size of the flexible package 10.

FIGS. 5, 6, 7, 8 and 11 each show a removable opening device. In contrast, the opening devices shown in FIGS. 1, 9 and 10 are not designed to be removable. In the present invention, by having an opening device which is not removable, the opening device may be used to cover and protect the articles contained within the interior compartment. On the other hand, by having an opening device which is removable, other uses of the opening device may be obtained, which are described in more detail below. If the opening device is not removable, it may contain an additional means for holding the opening device in a closed position after opening and possibly reattaching the opening device to the top surface. Examples of such holding means include, for example, a piece of tape which extends beyond the edge of the opening device, and other such means which will allow for closing of the opening device.

The opening device may also contain an opening aid. An opening aid provides a means for a consumer to grab the opening device 5 and open the opening device 5. The opening aid may be located within the opening devices, externally attached to the opening device or close to the opening device so that a consumer can grab the opening aid and/or the opening device. The opening aid should be adapted or sized to receive at least one human finger so that the consumer may insert their finger and grab the opening aid or opening device. In one configuration of the present invention, the opening aid is located close to or within the opening device. Ideally, the opening aid should be sized and shaped to receive two, three or four fingers of a person's hand so that the person's fingers can be positioned in the opening aid to provide a means for the user to grab and open the opening device. In one aspect of the present invention, the opening aid may be a tab attached to the opening device (not shown in figures).

Attention is directed to FIGS. 7-9 which all show possible configurations for the opening aid 77. In FIGS. 7 and 9, the opening aid 77 includes lines of weakness 78 which are of a size and shape which enable the consumer to insert their fingers into the flexible package 10, so that the opening device 5 can be placed between the consumer's fingers. The lines of weakness 78 are similar to the lines of weakness of the opening device 5. In FIG. 7, the lines of weakness 78 are configured in a crisscross shape. In FIG. 9, the lines of weakness 78 of the opening aid 77 are configured in a circular shape. It is noted that other shapes are within the scope of the present invention and can be used without departing from the present invention. Other possible shapes include, for example, square, rectangular, triangular, a Y-shape, an I-shape, a U-shape, a single line and the like. As a consumer attempts to insert their fingers into the opening aid 77, the lines of weakness 78 are caused to separate, allowing the consumer or user of the articles to easily insert their fingers through opening aid 77. In another possible configuration shown in FIG. 8, apertures 79 are located in a location on the top surface of the flexible package, within the opening device or closely adjacent the opening device. The apertures function as an opening aid 77 and are of sufficient size to allow a consumer or user of the articles within the flexible package to insert at least one finger into an aperture. It is noted that FIG. 8 shows two apertures; however, it is within the present invention to have a single aperture or more than two apertures. In addition, the apertures may be shapes other than the circular, as shown in FIG. 8. The aperture opening aid may also serve a second purpose, allowing any trapped air to escape the flexible package as the articles are loaded into the flexible package and the flexible package is sealed. Other possible shapes for the aperture may be used including square, rectangular, triangular, a Y-shape, an I-shape, a U-shape, a single line and the like, provided that a user can insert at least one finger into the opening aid.

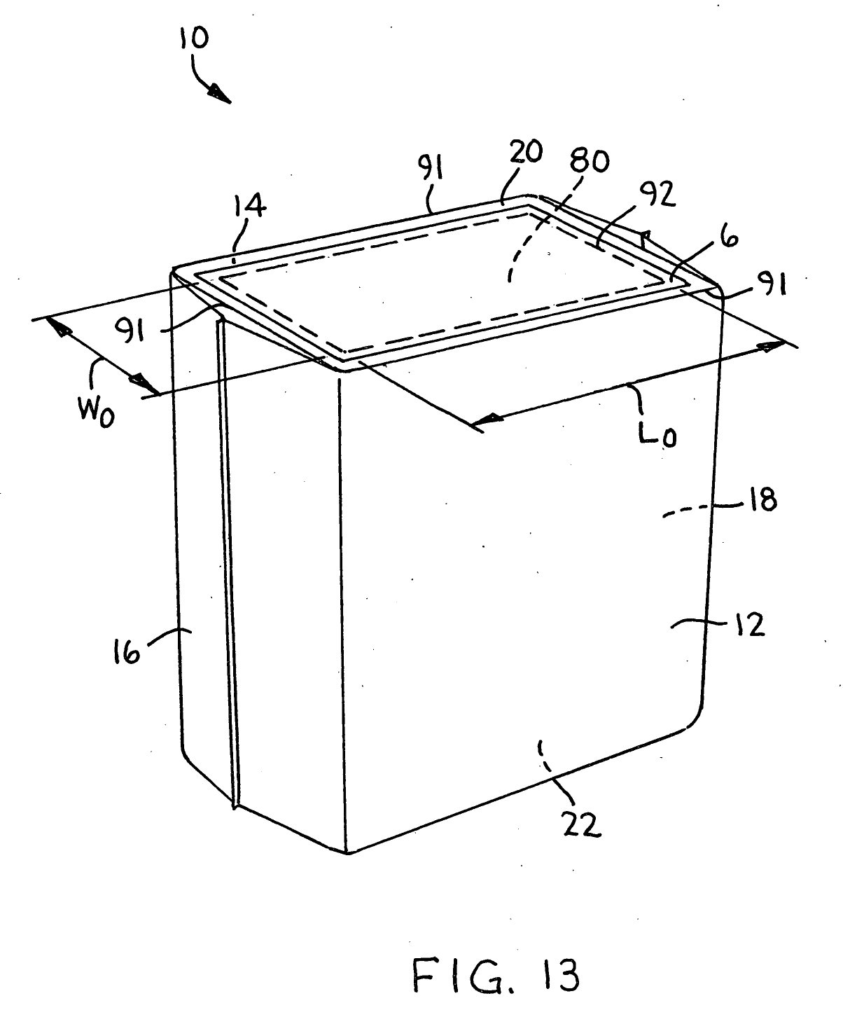

In an alternative configuration, the top surface 20 has an opening preformed therein, similar to the opening 80 shown in FIG. 13. In this configuration, the opening 80 is confined to the top surface such that there is a portion of the top surface located between the opening 80 and an edge 91 created by the direct or indirect connection of the top surface to the at least one side wall. The opening 80 has a parameter edge 92 and has a width Wo at some point along the length L of the opening which is between about 40% and about 99% of the width W of the top surface and a length Lo at some point along the width W of the opening which is between about 40% and about 99% of the length L of the top surface. In one embodiment, the opening 80 has a width Wo at its widest point along the length Lo of the opening 80 between about 50% and about 90% of the width W of the top surface. Desirably, the width Wo at its widest point along the length Lo of the opening device is between about 60% and about 80% of the width W of the top surface. In a similar manner, the opening 80 has a length Lo at its longest point along the width Wo of the opening 80 between about 50% and about 90% of the length L of the top surface. Desirably, the length Lo at its widest point along the width Wo of the opening device is between about 60% and about 80% of the length L of the top surface. The opening 80 allows a consumer to access the articles 26 within the interior compartment 24 of the flexible package. The opening 80 provides for easy access to articles within the interior compartment of the flexible package.

In the case where the flexible package has a preformed opening 80, the opening should be covered by a covering device 6, so that the articles 26 are retained within the interior compartment 24, prior to opening. In one aspect of the present invention, the covering device 6 may also function as an opening device 5 and may also contain opening aids as described above. For example, the covering device may be larger than the opening and may cover up to the entire top surface 20 of the package. Generally, the covering opening device should be slightly larger than the opening 80. The covering device may be secured to the top surface 20 using any means known to those skilled in the art and may be temporary or a permanent securing means. Examples of a securing means include, without limitation, adhesives, thermal bonds, ultrasonic bonds, stitching, spot welds and the like. Of these securing means, adhesives are generally desired, since the adhesive can be selected such that the covering device may be resecured to the top surface, thereby protecting the articles inside the interior compartment.

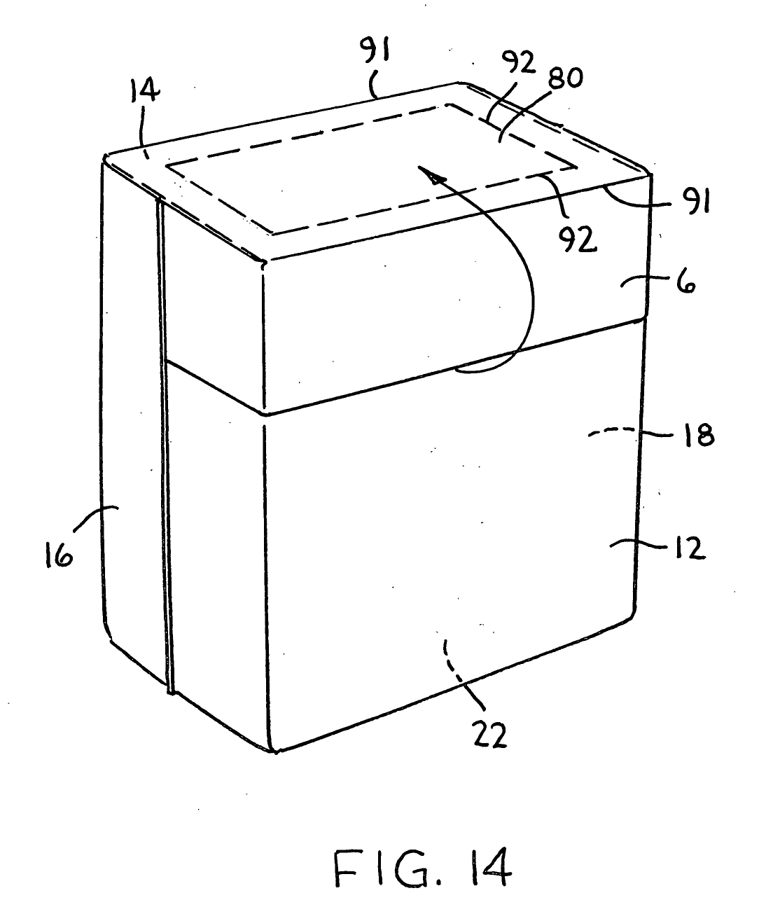

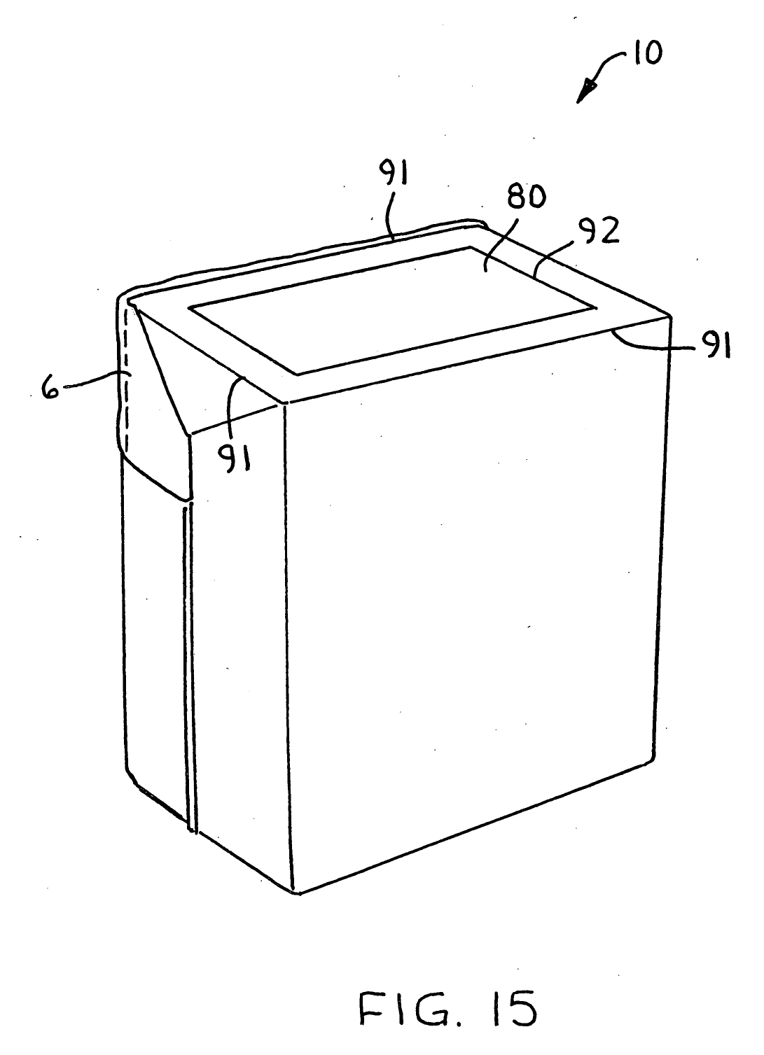

Another possible covering device 6 is shown in FIGS. 14 and 15. This covering device may be used whether the package has a preformed opening 80 or an opening device 5 shown in FIGS. 1, 2, and 5-9. The covering device is attached to one of the surfaces, generally the back 14, front 12 or top surface of the package 10 and is of sufficient size to cover the opening 80, the top surface 20 and at least a portion of the sides 16 and 18, the front 12 and the back 14 of the package 10. This covering device may be formed from the same material in which the package is constructed, or may be prepared from a different material.

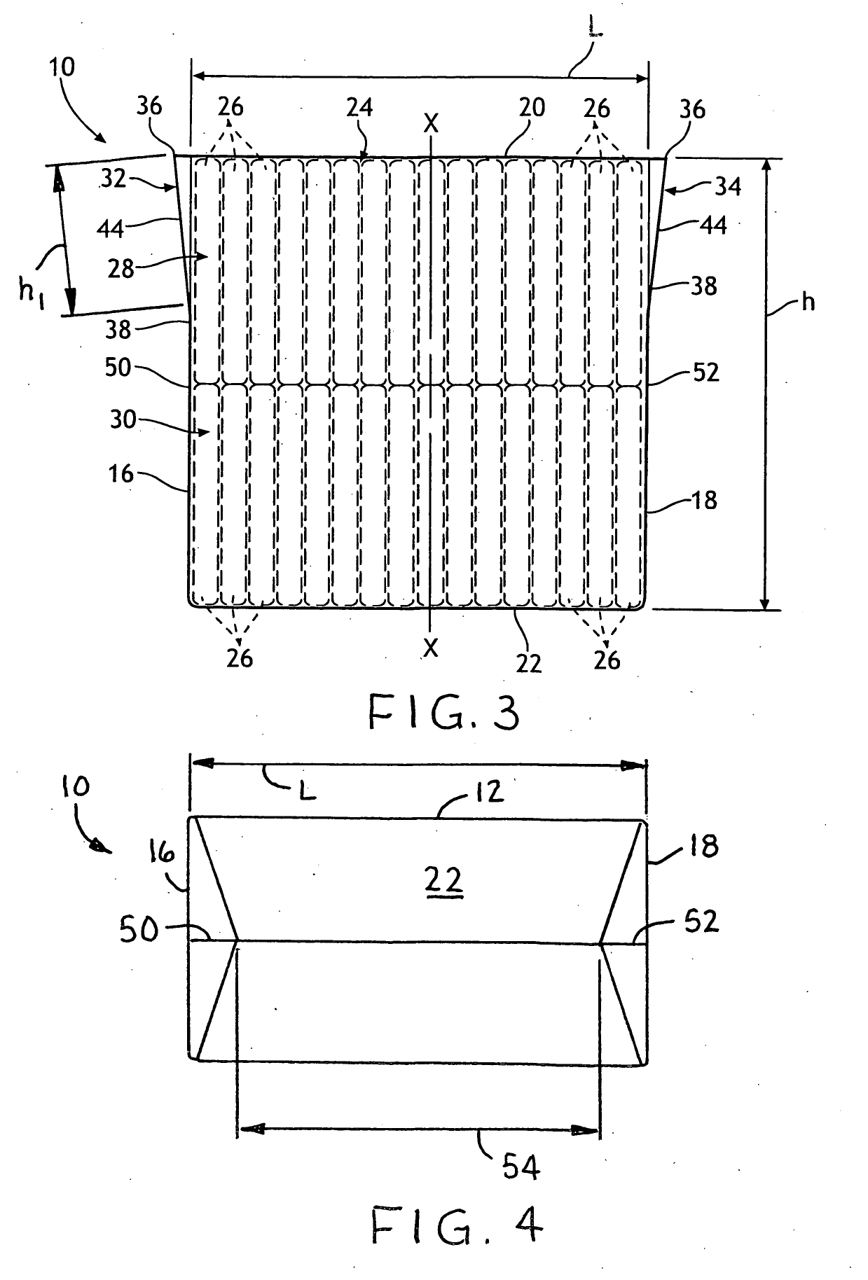

Referring now to FIGS. 1-3, the flexible package 10 is constructed with at least one gusset 32, and desirably, with a pair of gussets 32 and 34. By a “gusset” it is meant a member, for example a triangular member, capable of strengthening and/or enlarging the flexible package 10. The gusset 32 can be a separate piece of material or can be an extension of or integrally formed from the material from which the flexible package 10 is constructed. The gusset 32 can be viewed as a pocket, receptacle, cavity or opening. The one or two gussets, 32 or 32 and 34, are located in the top of the package 10 and are exposed to make them visible to the ultimate consumer. When two gussets 32 and 34 are present, they can be located on the opposite sides of the top wall 20, on opposite sides of the bottom wall 22 or one in the top wall 20 and one in the bottom wall 22 so as to provide a natural location where the consumer can easily grasp the package 10. The first gusset 32 is formed in at least a portion of the side wall 16 and the second gusset 34 is formed in at least a portion of the side wall 18. Each of the gussets 32 and 34 is shown as having a triangular configuration, although variations of the triangular shape can be employed. The actual configuration of the gussets 32 and 34 can be formed by folding the material from which the package 10 is constructed. Each of the gussets 32 and 34 has a first end 36 that can be aligned with the top wall 20 or can be slightly offset therefrom. Each of the gussets 32 and 34 has a second end 38 which is spaced away from the first end 36 and extends downward toward the bottom wall 22. The first end 36 represents the base of the triangular configuration of each of the gussets 32 and 34 and the second end 38 represents the apex of the triangular configuration. Each of the gussets 32 and 34 has a height h₁ that extends at least about 10% of the package height H (see FIG. 3). Desirably, each of the gussets 32 and 34 has a height h₁ that extends at least about 30% of said package height h. More desirably, each of the gussets 32 and 34 has a height h₁ that extends from between about 20% to about 90% of the package height H. The height h₁ of the gusset 32 or 34 can vary depending upon the width of the package 10. For example, as the width of a package 10 increases, the height h₁ of the gusset 32 or 34 may generally get bigger.

Each of the gussets 32 and 34 is aligned inward of a portion of one of the pair of opposing side walls 16 and 18 to form a pocket 40. Each pocket 40 has an internal panel (not shown) and an external panel 44. Each pocket 40 is formed by folding the material forming the package 10 such that the internal panel and the external panel 44 are joined together and extend diagonally downward from the opposite upper corners of the package 10 down to the second end 38. In FIG. 3, the front wall 12, the top wall 20 and the side wall 16 form a front upper corner 46 while the back wall 14, the top wall 20 and the side wall 16 form a back upper corner 48 (see FIG. 1). The internal and external panels are each joined at the corners 46 and 48 and have a common line of intersection that diverges diagonally downward and inward toward the second end 38. The function of the gussets 32 and 34 is to strengthen the upper region of the side walls 16 and 18 and to provide an enlarged area whereby the consumer can position one, two or more of his or her fingers so as to easily carry the package 10.

Referring now to FIGS. 1-4, the flexible package 10 also includes a pair of seals 50 and 52, each formed in the opposing side walls 16 and 18. The pair of seals 50 and 52 can be formed by a heat and pressure bond, by a thermal bond, by an ultrasonic bond, by adhesive or by another means known to those skilled in the art. The pair of seals 50 and 52 is present in the external panels 44 of the pockets 40 and each spans the entire height H of the package 10. Each of the pair of seals 50 and 52 extends from the first end 36 of one of the gussets 32 and 34 downward into the bottom wall 22. The pair of seals 50 and 52 can be aligned parallel to the central longitudinal axis of the side wall 16, if desired. In FIG. 4, one can see that the pair of seals 50 and 52 actually extends into and across a portion of the bottom wall 22. The distance that each of the pair of seals 50 and 52 extends across a portion of the bottom wall 22 can vary. Desirably, the pair of seals 50 and 52 will extend across at least about 10% of the length L of the bottom wall 22. The purpose of the pair of seals 50 and 52 is to secure the pair of side walls 16 and 18 together whereby the front wall 12, the back wall 14, the pair of side walls 16 and 18, and the top wall 20 create the internal compartment 24 which is open only at the bottom wall 22. The package 10 is designed to have the multiplicity of articles 26 inserted into it via the open bottom wall 22. After the articles 26 are positioned within the package 10, the bottom wall 22 will then be sealed.

Referring to FIG. 4, a bottom seal 54 may also be present and is formed in the bottom wall 22 after a plurality of articles 26 are placed into the internal compartment 24 of the package 10. Desirably, the articles 26 are compressed before being positioned within the internal compartment 24. Once the articles 26 are positioned with the package 10, the bottom wall 22 is sealed by any of the bonds described above with reference to the pair of seals 50 and 52. A heat and pressure bond works well for a polymeric film material. The bottom seal 54 cooperates with said pair of seals 50 and 52 to completely enclose the articles 26 within the package 10. By “completely enclose” it is meant that the plurality of articles 26 are surrounded on all sides by the material forming the package 10. The bottom seal 54 can be aligned parallel to the central transverse axis of the package 10, if desired.

The package 10 can be prepared from a single piece of material or can be prepared from multiple pieces of material. If multiple pieces of material are used, the individual pieces must be joined together using a suitable means. For example, the individual pieces may be joined by various conventional techniques, such as adhesive bonding, thermal bonding, ultrasonic bonding, welding, and so forth. In another embodiment, the panels are connected with mechanical fastening systems, such as sewing, stapling, riveting, and so forth. In one embodiment, the package 10 is formed from a continuous roll of material having a pre-formed gusset and perforations. Sealing of the two panels at the side seam, such as with heat and compression, which also causes the individual bags to separate during production.

In all instances, it is important that adequate welds or seals are produced at all locations. A combination of time, temperature, pressure, seal area and/or bag film materials may be used to accomplish an adequate seal as is known in the art. Seals are tested using standard industry methods, although seal strength requirements vary by individual specifications, depending on consumer, product and equipment needs. Strength tests are performed not only on the bag, but also on both side seams (including gusset area seals), as well as the perforations by methods known in the art. In some instances, an AMTS Sintech 1″ made by MTS Systems Corporation in Minneapolis, Minn. is used to test seal and perforation strength.

The package 10 may be made from any one of a wide variety of materials that are known in the art to be sufficiently flexible to accommodate the desired number of articles 26 and have sufficient strength to hold and contain the articles 26 without breaking and without excessive bulging or stretching of the material. Materials include, but are not limited to, polymeric plastic films, foils, paper, paper composites, knitted or woven fabrics, nonwoven fabrics and the like, or a combination thereof. Suitable materials can be made from polymeric materials such as polyethylene, polypropylene, polyester, nylon, and the like, as well as any combination thereof. In one embodiment, the material is a low density polyethylene (LDPE) film. In another embodiment, the material is a LDPE/LLDPE (linear low density polyethylene) film laminate. In yet another embodiment, the material is a LDPE/MDPE (medium density polyethylene) film laminate, a LDPE/HDPE (high density polyethylene) film laminate or the like. In another embodiment a polyethylene/polypropylene combination is used. In a specific embodiment, the material is a polyethylene film or film laminate having a thickness of between about 1 and about 5 mils (about 0.025 to 0.125 mm).

Referring to FIG. 3, the flexible package 10 may be filled with a plurality of articles 26. The articles 26 can be randomly or uniformly arranged within the package 10. Desirably, the articles 26 are placed within the interior compartment 24 of the package 10 in a uniform arrangement, in particular, arranged within the interior compartment 24 in one or more rows. In one configuration, as shown in FIG. 3, the articles 26 are arranged in at least one row 28 which extends from one side wall 16 to the other side wall 18. Other possible configurations include arranging the articles 26 in at least one row which extends from top surface 20 to the bottom surface 22 (not shown) or in at least one row which extends from the front wall 12 to the back wall 14 (also not shown). The number of rows of articles 26 contained within the interior compartment 24 is not critical to the present invention and is generally dependant on the size of the articles in relation to the size of the package 10. As is shown in FIG. 3, two rows of articles 28 and 30 are depicted with an upper row 28 being located above a lower row 30 within the interior compartment 24 of the package 10. Other arrangements of the articles could be used. For example, two rows could be arranged side-by-side, or more than two rows of articles could be present in the interior compartment 24. Likewise, the package 10 can contain within the interior compartment 24 two or more rows arranged along the lower portion of the package 10 and two or more rows located above the lower rows. For example, a package can contain two bottom rows and two vertical rows thereby forming an aggregate of four rows within the package. Another example would be a package containing two bottom rows and three vertical rows thereby forming an aggregate of six rows within the package. Any other conceivable arrangement may be used for the articles 26 contained within the package 10 without departing from the present invention.

Each row 28 and 30 can consist of a plurality of articles 26. As shown in FIG. 3, each of the rows 28 and 30 contains 16 articles. However, it should be readily apparent that the number of articles 26 contained within a given row may vary. For disposable absorbent articles, either wrapped with a wrapper material or unwrapped, the number of articles 26 enclosed within a single package 10 usually ranges from between about 3 to about 200. Generally, there are from about 5 to about 100 disposable absorbent articles in a given row. Particularly, there are from about 8 to about 50 disposable absorbent articles in a given row. The number of articles 26 in each row 28 and 30 can be the same or can differ. The articles 26 may be capable of being compressed. Desirably, each article 26 can be compressed by at least 10%, and desirably, by at least 20%. By compressing the articles which are capable of being compressed, the overall size of the package can be reduced, thereby providing a savings of package material and storage space. The articles 26 may be almost any type of article. One particular use of the flexible package of the present invention is to hold and dispense disposable absorbent personal care articles 26. A disposable absorbent personal care article is a product that is primarily designed and constructed to absorb human discharge, such as urine, menses and/or fecal matter. The disposable absorbent article is a product that is designed for a single use before it is discarded and is not intended to be laundered and reused. Examples of disposable absorbent articles 26 include infant diapers, training pants, sanitary napkins, feminine pantiliners and pads, tampons, adult incontinence garments, such as pads, briefs and undergarments, as well as other disposable absorbent products.

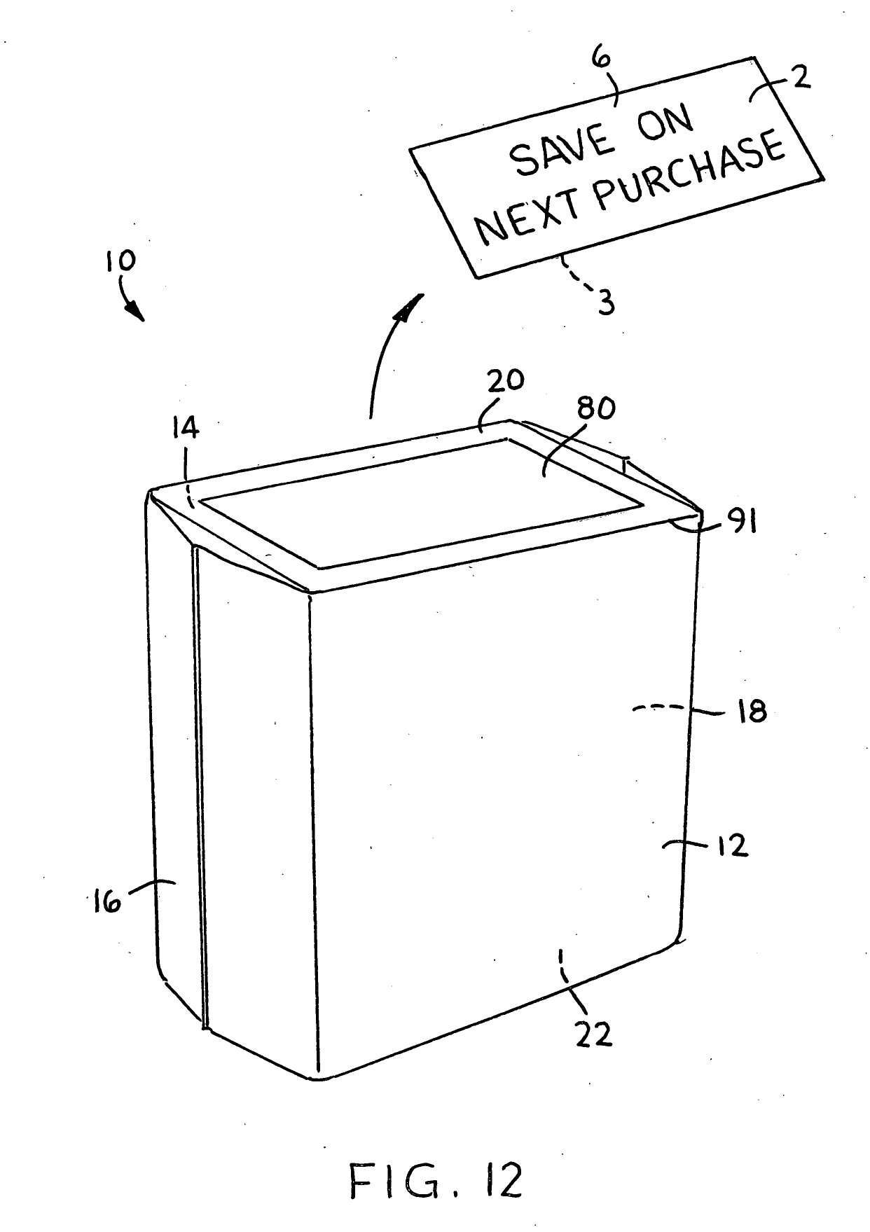

Another advantage of the opening device of the present invention is that the opening device may be printed with information which may be conveyed to the consumer. As is shown in FIG. 12, the opening device has a front side 3 and a back side 2. The front side 3 is the side of the opening device in which the consumer or user sees when the package is closed and the back side 2 of the opening device 5 is viewable when the opening device 5 is opened. Information which may be provided on the back side 2 of the opening device 5, includes, for example, an inspirational message, a promotional offer such as coupons for future purchase of a similar or different product and points towards an offer such as free products, a proof of purchase, information regarding the absorbency and size of the product contained within the interior compartment, a guide to other similar products within a given product line, a thank you message and the like.

In addition, the package of the present invention may be provided with other features, including windows or clear panels to view the product inside the interior compartment.

The dimensions of the package 10 may vary, depending on the type and number of articles 26 being packaged. Generally, the package 10 has a rectangular structure, although the invention is not so limited. In one embodiment, the package is about 16 cm in height, about 22 cm in length, and about 10 cm in width (depth). Of course these dimensions can be varied, depending on the type, amount and size of the articles 26 which are placed in the package 10, without departing from the scope of the present invention. The base or bottom surface of the package should be of a sufficient width and length so that the base or bottom surface will support the height of the package, although this is not required of the present invention. Although not shown in the figures herein, the corners of the packages, such as the corner defining the transition from the top section 20 to the front or back surface 12 or 14, are slightly curved. Other conventional construction features of the bag 10 should be understood by those skilled in the art and will not be discussed in detail herein. Essentially, any type of flexible bag known in the art can be used, and further information on material choices is given below.

If the package of the present invention is filled with absorbent personal care articles, generally the height of the flexible package should be at least about ¼ of the width W. Usually, the height of the package is at least about % of the width and often greater than or equal to the width. Of course, the size and ratio of the height to width of the package may be adjusted according to the types of articles stored and dispensed from the package.

In the present invention, the opening device may be highlighted to direct a user's attention to the location where the flexible package may be opened. Highlighting may be accomplished by various means, including, for example, coloring the opening device a color which does not appear on the remainder of the package, outlining the opening device with color which attracts attention, e.g. orange or fluorescent type colors, highlighting the lines of weakness or providing other indicia to clearly indicate the location of the opening device. In addition, instructions to the user as to how to open the flexible package may be located on the opening device.

Finally, the opening device and/or opening may be formed on the package in a variety of ways. It may be formed prior to the formation of the flexible package or after the flexible package is formed. Both the opening device and/or opening may be formed by die-cutting the line of weakness or opening into the packaging material while the material is in a flat configuration. Other methods include folding the packaging material in half prior to forming and cutting or perforating the material from the edge where the packaging material is connected and through both of the layers of the folded material. The latter method helps ensure that the opening is symmetrical.

The flexible package of the present invention may optionally be printed or otherwise provided with graphics to convey to the consumer the contents of the flexible package. The graphics on the package, if present, may optionally be coordinated with the contents of the package.

Although the present invention has been described with reference to various embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A flexible package comprising
a) a top surface, a bottom surface and at least one side wall; said top surface and said bottom surface each being directly or indirectly connected to said at least one side wall and form an interior volume capable of containing a plurality of articles, said top surface has a width and a length; and
b) an opening device located on said top surface and said opening device comprises at least one line of weakness, said opening device being confined to the top surface such that there is a portion of the top surface located between the opening device and an edge of the top surface created by the direct or indirect connection of the top surface to the at least one side wall, said opening device comprises a width at some point along the length of the opening device which is between about 40% and about 99% of the width of the top surface and a length at some point along the width of the opening device which is between about 40% and about 99% of the length of the top surface.

2. The flexible package of claim 1, wherein the opening device comprises a continuous line of weakness.

3. The flexible package of claim 2, wherein the line of weakness comprises an oval or bilobal shape.

4. The flexible package of claim 1, wherein the opening device comprises a first, a second, a third and a fourth line of weakness, wherein said first line of weakness has a first end and a second end, said third line of weakness comprises a first end and a second end, said second line of weakness joins the first end of the first line of weakness to the first end of the third line of weakness and said fourth line of weakness joins the second end of the first line of weakness to the second end of the third line of weakness.

5. The flexible package of claim 4, wherein the first line of weakness is parallel to the third line of weakness and the second line of weakness is parallel to the fourth line of weakness and the first and third lines of weakness are perpendicular to the second and fourth line of weakness.

6. The flexible package of claim 4, wherein said second line of weakness is joined to the first end of the first line of weakness by a first connecting line and said second line of weakness is joined to the first end of the third line of weakness by a second connecting line, and said fourth line of weakness is joined to the second end of the third line of weakness by a third connecting line and said fourth line of weakness is joined to the second end of the first line of weakness by a fourth connecting line.

7. The flexible package of claim 6, wherein each connecting line comprises a line of weakness.

8. The flexible package of claim 7, wherein each connecting line is a line independently selected from the group consisting of a straight line, a curved line, a zig-zag line and a combination thereof.

9. The flexible package of claim 8, wherein each connecting line is a curved line.

10. The flexible package of claim 4, wherein said second line of weakness has a first end and a second end and the said fourth line of weakness has a first end and a second end, the first end of the second line of weakness is the first end of the first line of weakness, the second end of the second line of weakness is the first end of the third line of weakness, the first end of the fourth line of weakness is the second end of the third line of weakness and the second end of the fourth line of weakness is the second end of the first line of weakness.

11. The flexible package of claim 10, wherein the first line of weakness is parallel to the third line of weakness and the second line of weakness is parallel to the fourth line of weakness and the first and third lines of weakness are perpendicular to the second and fourth line of weakness.

12. The flexible package of claim 1, wherein the opening device comprises a first line of weakness, a second line of weakness and a third line of weakness, each line of weakness comprising a first end and a second end, wherein the first end of the first line of weakness is adjacent the first end of the second line of weakness, and the second end of the second line of weakness is adjacent the first end of the third lines of weakness.

13. The flexible package of claim 12, wherein the second end of the third line of weakness is adjacent the second end of the first line of weakness.

14. The flexible package of claim 12, wherein at least one of the lines of weakness is non-linear.

15. The flexible package of claim 12, wherein the opening device remains attached to the flexible package.

16. The flexible package of claim 1, wherein the at least one side wall comprises a front side wall, a back side, a first side wall and a second side wall, wherein the front side wall is opposed to the back side wall and first side wall is opposed to the second side wall, the front side wall and the back side wall are directly or indirectly connected to the first and second side walls.

17. The flexible package of claim 1, wherein the opening device further comprising a means for grabbing the opening device.

18. The flexible package of claim 17, wherein the means for grabbing the opening device comprises slits, apertures or perforations, each of which being of a size to receive at least one human finger and are located in or adjacent the opening device.

19. The flexible package of claim 17, wherein the means for grabbing the opening device comprises a tab attached directly or indirectly to the opening device.

20. The flexible package of claim 1, further comprising a plurality of articles placed within the interior volume.

21. The flexible package of claim 1, wherein the opening device is removable from the top surface of the flexible package.

22. The flexible package of claim 1, wherein the opening device while on the package has an exterior side and an interior side, wherein one of the sides comprises a message to a user of the articles contained within the flexible package.

23. The flexible package of claim 22, wherein the message comprises information regarding the type of article contained within the flexible package, words of inspiration, a coupon or a proof of purchase.

24. The flexible package of claim 23, wherein the message is on the interior side of the opening device.

25. The flexible package of claim 24, wherein the opening device is removable from the top surface of the flexible package.

26. The flexible package of claim 20, wherein the articles within the interior volume are oriented such that they are perpendicular to the top surface.

27. The flexible package of claim 26, wherein the articles are arranged in at least one row.

28. The flexible package of claim 26, wherein the plurality of articles are arranged in at least two rows, each row comprising at least a portion of the plurality of articles.

29. The flexible package of claim 20, wherein the articles comprise an absorbent personal care article.

30. The flexible package of claim 29, wherein the absorbent personal care articles comprises a sanitary napkin, an incontinence pad, a diaper, a training pant, or an incontinence undergarment.

31. The flexible package of claim 1, wherein the opening device comprises a width at some point along the length of the opening device which is between about 50% and about 90% of the width of the top surface.

32. The flexible package of claim 31, wherein the opening device comprises a width at some point along the length of the opening device which is between about 60% and about 80% of the width of the top surface.

33. The flexible package of claim 1, wherein the opening device comprises a length at some point along the width of the opening device which is between about 50% and about 90% of the length of the top surface.

34. The flexible package of claim 29, wherein the opening device comprises a length at some point along the width of the opening device which is between about 60% and about 80% of the length of the top surface.

35. The flexible package of claim 1, wherein said at least one side wall comprises a front side wall, a back side wall, a first side wall and a second side wall, wherein the front side wall is opposed to the back side wall and first side wall is opposed to the second side wall, the front side wall and the back side wall are directly or indirectly connected to the first and second side walls, the opening device comprises a first, a second, a third and a fourth line of weakness, wherein said first line of weakness has a first end and a second end, said third line of weakness comprises a first end and a second end, said second line of weakness joins the first end of the first line of weakness to the first end of the third line of weakness and said fourth line of weakness joins the second end of the first line of weakness to the second end of the third line of weakness, the flexible package comprises a plurality of articles placed within the interior volume, the opening device comprises a width at some point along the length of the opening device which is between about 60% and about 80% of the width of the top surface and the opening device comprises a length at some point along the width of the opening device which is between about 60% and about 80% of the length of the top surface.

36. The flexible package of claim 35, wherein the opening device is removable from the top surface of the flexible package.

37. The flexible package of claim 36, wherein the articles within the interior volume are oriented such that the articles are perpendicular to the top surface.

38. The flexible package of claim 37, wherein the articles are arranged in at least one row.

39. The flexible package of claim 37, wherein plurality of articles are arranged in at least two rows, each row comprising at least a portion of the plurality of articles.

40. The flexible package of claim 1, wherein the line of weakness comprises perforations.

41. A flexible package comprising
a) a top surface, a bottom surface and at least one side wall; said top surface and said bottom surface each being directly or indirectly connected to said at least one side wall and form an interior volume capable of containing a plurality of articles, said top surface has a width and a length;
b) an opening located on said top surface and said opening being confined to the top surface such that there is a portion of the top surface located between the opening and an edge of the top surface created by the direct or indirect connection of the top surface to the at least one side wall, said opening comprises a width at some point along the length of the opening which is between about 40% and about 99% of the width of the top surface and a length at some point along the width of the opening which is between about 40% and about 99% of the length of the top surface;
c) a covering means for the opening.

42. The flexible package of claim 41, wherein the covering means comprises a sticker.

43. The flexible package of claim 42, wherein the sticker comprises an adhesive which allows the package to be resealed after opening.

44. The flexible package of claim 41, wherein the covering means comprises a flap.
